(19) United States
(12) Patent Application Publication
(10) Pub. No.: US 2014/0088452 A1
(43) Pub. Date: Mar. 27, 2014

(54) STRESS EVALUATION APPARATUS, STRESS EVALUATION METHOD AND STRESS EVALUATION PROGRAM

(71) Applicants: A SCHOOL CORPORATION KANSAI UNIVERSITY, Osaka (JP); DENSO CORPORATION, Kariya-city (JP)

(72) Inventors: Takahiro ISHIKAWA, Nagoya-city (JP); Kentaro KOTANI, Suita-city (JP); Satoshi SUZUKI, Toyonaka-city (JP); Takafumi ASAO, Yamatokoriyama-city (JP)

(73) Assignees: A SCHOOL CORPORATION KANSAI UNIVERSITY, Osaka (JP); DENSO CORPORATION, Kariya-city (JP)

(21) Appl. No.: 14/032,731

(22) Filed: Sep. 20, 2013

(30) Foreign Application Priority Data
Sep. 24, 2012 (JP) 2012-209761

(51) Int. Cl. A61B 5/0488; A61B 5/00

(52) U.S. Cl. CPC A61B 5/0488; A61B 5/6813; A61B 5/7271
USPC 600/546

(57) ABSTRACT

A stress evaluation apparatus, a stress evaluation method, and a program for evaluating stress received by an operator during when an in-vehicle device is operated by an upper limb are provided. The apparatus includes a detection sensor, a calculation process portion and an evaluation portion. The detection sensor detects a muscle potential of a measuring object muscle of the operator. The calculation process portion receives time series information of the muscle potential to calculate strength information. The evaluation portion compares the strength information with an evaluation standard to make an evaluation of stress of the operator. The method includes detecting a muscle potential of a measuring object muscle, receiving time series information of the muscle potential to calculate strength information, and comparing the strength information with an evaluation standard to evaluate stress. A medium storing a computer-executable program that causes a computer to perform the above-described method is provided.

### CROSS REFERENCE TO RELATED APPLICATION

This application is based on Japanese Patent Application No. 2012-209761 filed on Sep. 24, 2012, the disclosure of which is incorporated herein by reference.

### TECHNICAL FIELD

The present disclosure relates to a stress evaluation apparatus, a stress evaluation method and a stress evaluation program for evaluating stress of a driver. Especially, the present disclosure relates to the stress evaluation apparatus, the stress evaluation method, and the stress evaluation program when an in-vehicle device is operated by an upper limb.

### BACKGROUND

Conventionally, Japanese Patent No. 4433739, corresponding to U.S. Pat. No. 7,467,010B, proposes a stress evaluation apparatus which measures a muscle potential of a masseter muscle to objectively evaluate mental stress such as a riding comfortability or handling. The mental stress to be evaluated may be stress during working such as stress of driving a vehicle or the like.

A reason for measuring the muscle potential of the masseter muscle is that a worker (a test subject) in performing a subject work produces a muscle power by contracting a muscle for supporting a head of the worker to keep posture of the worker in a case where an external force is applied to the head by the subject work. It is assumed that the muscle potential of the masseter muscle well reflects the mental stress such as the riding comfortability or the handling. Thus, it is considered that, when the muscle potential of the masseter muscle is measured, the mental stress is objectively measured.

### SUMMARY

It is an object of the present disclosure to provide a stress evaluation apparatus, a stress evaluation method and a program for objectively evaluating stress that an operator receives when the operator operates various in-vehicle devices that include in-vehicle devices operated by an upper limb of an operator.

According to a first example of the present disclosure, a stress evaluation apparatus is provided. The stress evaluation apparatus includes a detection sensor, a calculation process portion and an evaluation portion. The detection sensor detects a muscle potential of a measuring object muscle of the operator. The calculation process portion receives time series information of the muscle potential, which is detected by the detection sensor, and calculates strength information of the muscle potential based on the received times series information of the muscle potential. The evaluation portion compares the strength information with a predetermined evaluation standard, and evaluates stress that the operator receives in an in-vehicle device operation. The measuring object muscle includes a muscle that connects an upper limb and a spine, or another muscle that connects the upper limb and an anterior chest wall and that connects the upper limb and a lateral chest wall. The in-vehicle device is operated by the upper limb.

According to a second example of the present disclosure, a stress evaluation method is provided. The stress evaluation method includes detecting a muscle potential of an measuring object muscle of an operator by a detection sensor, receiving time series information of the muscle potential detected by the detection sensor to calculate strength information of the muscle potential based on the received time series information of the muscle potential, and comparing the strength information with a predetermined evaluation standard to make a evaluation of stress that the operator receives when an in-vehicle device is operated by the operator.

According a third example of the present disclosure, a non-transitory tangible computer readable storage medium storing a computer-executable program that causes a computer to perform the above-described method is provided.

According to the above stress evaluation apparatus, the stress evaluation method and the program, the stress that the operator receives in driving is objectively evaluated when various in-vehicle devices are operated by an upper limb of the operator.

### BRIEF DESCRIPTION OF THE DRAWINGS

The above and other objects, features and advantages of the present disclosure will become more apparent from the following detailed description made with reference to the accompanying drawings. In the drawings:

FIG. 1 is a block diagram illustrating a stress evaluation apparatus;

FIG. 2 is a block diagram illustrating a navigation device;

FIG. 3 is a flowchart of an evaluation process executed in the stress evaluation apparatus;

FIG. 4 is a graph illustrating a relationship between an operation performed by a user and a standard score in a demonstration experiment.

### DETAILED DESCRIPTION

Recently, as a result of an introduction of various information communication technologies to a vehicle, a vehicle information device becomes high and multi functional so that a human-machine interface (HMI), which connects a driver and a vehicle, becomes complicated.

Although the above device increases safety and convenience of the vehicle, it is required to well consider so as not to make distraction by excessively directing attention of the driver to an operation of the device.

A stress evaluation by measuring a muscle potential of a masseter muscle is related to mental stress such as a riding comfortability or handling.

However, the stress evaluation by measuring the muscle potential of the masseter muscle can not evaluate how an operator or a driver (hereinafter, referred to as the operator) receives stress that is provided by various stress factors from an external driving circumstance or the like when the operator executes each operation in driving. When the operator uses the vehicle with the above vehicle information device, the operator needs to integrally operate various in-vehicle devices which are operated by an upper limb.

An embodiment according to the present disclosure will be described below with reference to drawings.

(Overall Configuration)

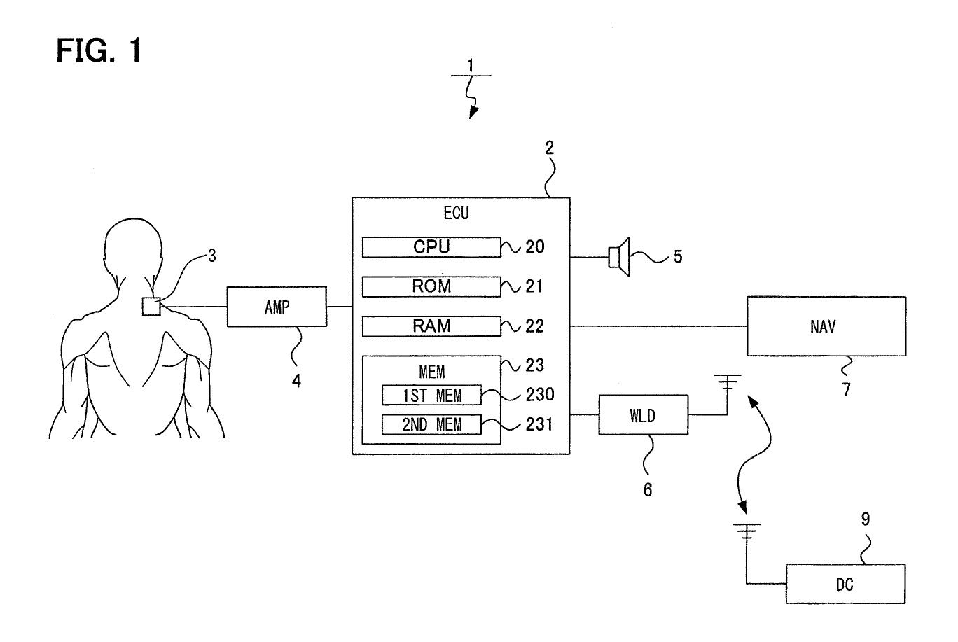

A stress evaluation apparatus 1 according to the present embodiment includes an electronic control unit (ECU) 2, an active electrode 3, an amplifier 4, a speaker 5 and a wireless device 6 as described in FIG. 1.

The ECU 2 is mounted on a vehicle and includes a computer having a CPU 20, a ROM 21, a RAM 22 or a memory 23. The ECU 2 executes a process including an evaluation process or the like. The active electrode 3 (corresponding to a detection sensor in the present disclosure) is pasted on a right shoulder of the operator and on a position where the muscle potential of a trapezius muscle is detectable since it is assumed to be a vehicle with a right-hand drive in the present embodiment Incidentally, the trapezius muscle corresponds to a measuring object muscle.

The amplifier 4 amplifies the muscle potential detected by the active electrode 3 and outputs the amplified muscle potential to the ECU 2. The speaker 5 outputs a warning sound according to a result in the evaluation process.

The wireless device 6 is mounted on the vehicle and wirelessly communicates with a data center 9. The navigation device 7 is placed at an instrumental panel of the vehicle at a substantially middle position between a driver's seat and a passenger's seat. The navigation device 7 can execute route guidance. The navigation device 7 communicably connects to the ECU 2.

(Navigation device 7)

Next, the navigation device 7 will be described particularly.

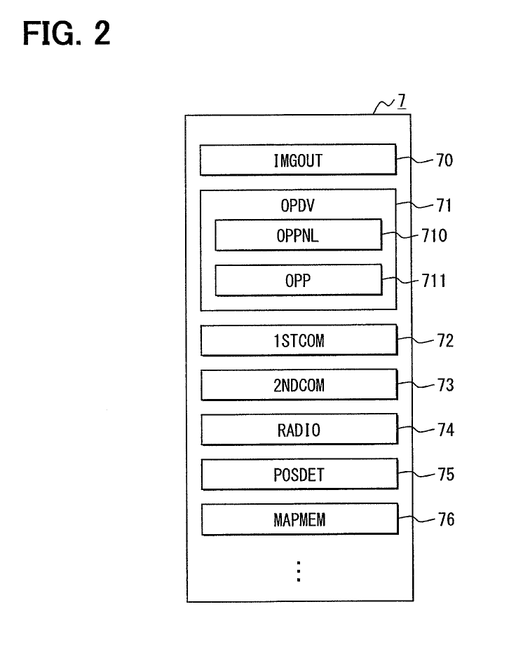

The navigation device 7 includes, for example, an image output device 70, an operation device 71, a first communication interface 72, a second communication interface 73, a radio receiver 74, a present position detection device 75, or a map information memory 76 as described in FIG. 2. The image output device 70 displays various screen images. An operation panel 710 is arranged on the image output device 70 and provides a transparent touch panel. The operation device 71 includes an operation portion 711, which is arranged around a screen of the image output device 70 and includes an operation button or the like. The first communication interface 72 communicates to the ECU 2. The second communication interface 73 communicates to a mobile phone. The radio receiver 74 receives a radio wave.

In addition, the navigation device 7 can execute, for example, functions described below besides conventional route guidance. The navigation device 7 executes following functions: a radio channel tuning function, a telephone number input function, a map scroll function. Firstly, the radio channel tuning function will be explained. The radio channel tuning function displays a channel tuning image on the image output device 70. The channel tuning image includes multiple simulated tuning buttons for selecting a radio station whose radio wave is received. In a case where a user operates an operation panel 710 displaying the tuning button, the radio station is selected and the radio receiver 74 receives a radio wave of the selected radio station so that sound is outputted through the speaker 5. The tuning button corresponds to a radio station. The radio station indicates the selected tuning button.

Next, the telephone number input function will be explained. The telephone number input function displays an input image on the image output device 70. The input image includes, for example, multiple simulated numeric buttons (i.e., multiple buttons representing numbers) for inputting a telephone number, or the like. In a case where the user operates a position on the operation panel 710 corresponding to each of the numeric buttons according to a telephone number that the user will input, information related to the telephone number is inputted to the navigation device 7.

The information related to the inputted telephone number is utilized for, for example, searching a destination in a route search or identifying a telephone number when a phone call is made with the mobile phone after the information is sent to the mobile phone connected to the navigation device 7.

Next, the map scroll function will be explained. A map image is stored in the map information memory 76 for route guidance or the like. In a case where a part of the map image is displayed on the image output device 70, when the operation device 71 is operated, the map image is scrolled according to the operation.

Incidentally, the above functions are examples of functions of the navigation device 7.

(Memory 23)

Next, the memory 23 will be described particularly.

The memory 23 has at least two memory portions for storing information as described in FIG. 1. One of the memory portions is a first memory portion 230. The first memory portion 230 stores score relation information for calculating a standard score when the user operates the navigation device 7.

The other of the memory portions is a second memory portion 231. The second memory portion 231 stores evaluation standard information, received from the data center 9. The radio tuning function corresponds to a radio tuning operation, the telephone number input function corresponds to the telephone number input operation, and the map scroll function corresponds to a map scroll operation. In a case where the user executes each of the above described operations (i.e., the radio tuning operation, the telephone number input operation, and the map scroll operation) in driving, the muscle potential that is inputted through the active electrodes 3 is detected, and a root mean square (RMS) value is calculated based on the muscle potential. In the first memory portion 230, information related to a RMS mean value and a RMS standard deviation is stored as the score relation information.

The RMS value of the muscle potential is calculated as described below. Data of the muscle potential outputted from the active electrode 3 are sampled for a predetermined period when each of the operations is performed. The RMS value is calculated based on a total number of samplings. The RMS mean value denotes a mean value of multiple RMS values, which are obtained though multiple executions of each of the operations.

In addition, the RMS standard deviation denotes a standard deviation calculated from the multiple RMS values. A manner to obtain the score relation information is not limited to this manner. Thus, the score relation information may be generated in a vehicle or may be generated separately by performing various operations according to an instruction from the ECU 2, or the like.

In a case where the score relation information is generated in each vehicle, the operator pastes the active electrode 3 at a position where the muscle potential of the right trapezius muscle is measurable. The operator instructs the ECU 2 to execute instructions. According to instructions from the ECU 2, the operator performs various operations of the operation device 71 of the navigation device 7.

Since the RMS value is calculated in ECU 2 in each of the operations, when predetermined number of the RMS values are sampled, the RMS mean value and the RMS standard deviation may be calculated as the score relation information, and the first memory portion 230 of the memory 23 may store the score relation information.

The second memory portion 231 stores the evaluation standard information which provides an evaluation standard to evaluate stress of the operator. The evaluation is based on the standard score, which is calculated in the evaluation process. The standard score corresponds to strength information according to the present disclosure.

The evaluation standard information is calculated according to statistical values (e.g., a mean value or a standard deviation) of standard scores of the RMS values, which are obtained from various persons that perform each of the operations. The standard score of RMS values is obtained from each of the above described operations and a navigation operation, which is a prohibited operation in driving, with using a test track or the like in advance.

The evaluation standard information is stored in the data center 9 and is continuously updated. In the present embodiment, the information stored in the second memory portion 231 is updated in each time when the wireless device 6 receives the evaluation standard information from the data center 9.

In the present embodiment, levels 1 to 3 are determined according to a difference between the evaluation standard information and the standard score. The level 1 is allocated in a case where the difference between the evaluation standard information and the standard score is small, and the level 3 is allocated in a case where the difference is large. The level 1 is evaluated as no problem. The level 2 is evaluated that a caution is required. The level 3 is evaluated as danger (i.e., similar to the prohibited operation).

(Evaluation Process)

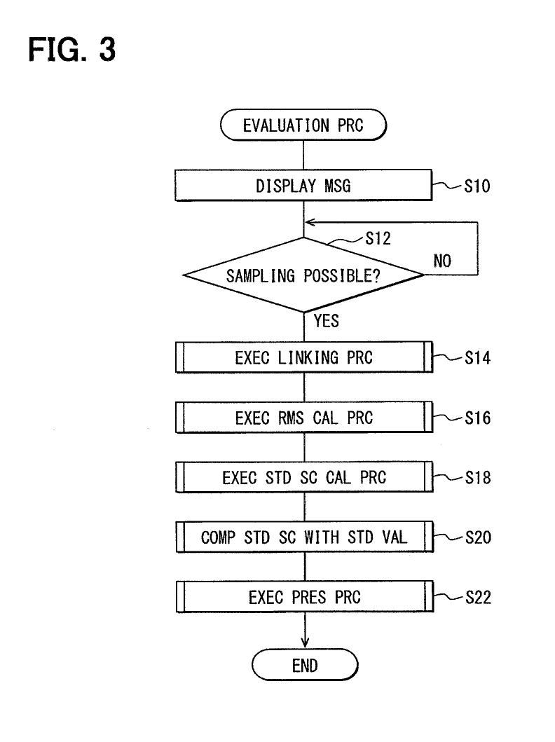

Next, the evaluation process executed in the ECU 2 will be described with reference to FIG. 3.

The evaluation process is repeated during a vehicle operation. In a case where the evaluation process starts, a message is displayed on the image output device 70 at step S10. The message demands that the active electrode 3 be pasted at a position where the muscle potential of the right trapezius muscle is measurable, and that a predetermined movement be performed in order to output the muscle potential through the active electrode 3 (at step S10).

It is determined at step S12 that the active electrode 3 is pasted by the operator and can measure the muscle potential. In a case where the muscle potential is measurable through the active electrode 3 (corresponding to “YES” at step S12), information representing the muscle potential is inputted through the active electrode 3 to the ECU 2 at predetermined sampling intervals.

In a case where the muscle potential is not measurable (corresponding to “NO” at step S12), the process waits and urges the operator to correctly paste the active electrode 3 again. At step S14, a linking process is executed and information, which is processed by the linking process, is stored into the RAM 22. In the linking process, information with regard to the muscle potential detected by the active electrode 3 is associated with time information.

At step S16, the RMS value is calculated based on the muscle potential information stored in the RAM 22. The muscle potential information is time series information associated with time information from a predetermined time ago to a present.

At step S18, by referring to the RMS mean value and the RMS standard deviation, which are stored in the first memory portion 230 of the memory 23, the standard score is calculated. Therefore, the stress that the operator feels is calculated as an objective value, which is the standard score.

Then, at step S20, the evaluation standard information, stored in the second memory portion 231 of the memory 23, and the standard score, calculated at step S18, are compared each other. It is determined whether the standard score corresponds to either of the levels 1 to 3. That is, the above process evaluates the stress of the operator.

At step S22, based on an result of an evaluation at step S20, a presentation process is executed in a case where it is evaluated that the standard score corresponds to the level 2 or the level 3. In the presentation process, according to the present embodiment, an operation of the navigation device 7 is totally prohibited and a notice is given through the speaker 5 in a case where the standard score corresponds to the level 3, for example. In the case where the standard score corresponds to the level 2, an operation (e.g., the input operation of the phone number) which causes an especially high stress among operations with regard to the navigation device 7 is prohibited and a notice is given through the speaker 5. On the other hand, in the case where the standard score corresponds to the level 1, it may be permitted to execute a prohibited operation. An operation is not prohibited and a notice is not given, especially.

In a case where the process at step S22 ends and when the vehicle is in operation, the process after step S14 is executed again. When the vehicle is not in operation, the process ends.

(Result of Experiment)

Next, an experiment will be explained so as to demonstrate whether a mental stress degree of the operator is objectively estimated by measuring the muscle potential of the trapezius muscle.

2.1 Experimental Participants

Nineteen men participated in the experiment. They have driver's licenses and are right handed. They drive a right-hand vehicle more than several times every month. Their average age is 21.7 and a standard deviation of their average age is 0.57. Their average year of driving experience is 2.79 and a standard deviation of their average years of driving experience is 1.28.

2.2 Experimental Task and Instruction

The experimental participants simultaneously performed (1) a driving task with a driving simulator (DS), (2) a navigation operation task, and (3) a multi-modal stimulus detection task. The multi-modal stimulus detection task corresponds to a stimulus detection task. The driving task corresponds to a primary task. In the navigation operation task, the experimental participant watches and operates a touch-panel vehicle information device (a navigation device) with one's hand. The navigation device is placed on a dashboard of the driving simulator. The experimental tasks are listed in order of descending priorities: the driving task, the navigation operation task, the stimulus detection task. The experimental participants were instructed the above priority.

2.2.1 Driving Task

In the driving task, the experimental participant followed a leading vehicle with keeping a distance between the leading vehicle and a subject vehicle without reeling sideways with using the DS. The distance which is enough for safety to the experimental participant was kept. The leading vehicle drives on a middle driving lane of an express way with repeated S-shape curves having three lanes each way. The leading vehicle runs 80-85 kilometers per hour.

2.2.2 Navigation Operation Task

There are fours kinds of operations in the navigation operation task. Timing to start operation was instructed by sound. The operation instructions were given through a small monitor, which is separated from the screen of the navigation device.

Following, the four kinds of operations will be explained.

A. No Operation of Navigation Device

This operation corresponds to a control condition. Any navigation operation is not performed.

B. Map Scroll

In this operation, the screen of the navigation device is touched and scrolled twice according to an arrow direction displayed on the small monitor. The number of times of a standard image screen transition is twice. The number of times of touch with the screen of the navigation device is three times.

C. Radio Channel Tuning

In this operation, two radio stations which are displayed on the small monitor are tuned in order. The number of times of a standard image screen transition is five times. The number of times of touch is six times.

D. Telephone Number Input

In this operation, a telephone number which is displayed on the small monitor is inputted and a destination is set. The number of times of a standard screen image transition is five times. The number of times of touch is fourteen times.

2.2.3 Multi-Modal stimulus Detection Task

In this experimental task, either of visual stimulus, tactile stimulus or auditory stimulus is given for 300 milliseconds in random order. The experimental participant immediately presses a button placed at a gripper of a steering wheel when the experimental participant feels the stimulus.

In a case where the experimental participant responds to the stimulus within less than 100 milliseconds, it is regarded as an invalid trial. In a case where the experimental participant responds to the stimulus form 100 milliseconds to 2000 milliseconds, it is regarded as a valid trial. In a case where the experimental participant does not respond to the stimulus within 2000 milliseconds, it is regarded as a missed trial. Herein, the trial denotes a push of the button. After one trial, a rest time with a random length for between 2000 milliseconds and 4000 milliseconds and then the next stimulus is given.

2.3 Experimental Procedure

Only the DS driving is performed as a single task (for about three minutes). Only a stimulus detection is performed as a single task (for about three minutes). The DS driving and the navigation device are simultaneously performed as a dual task (for about three minutes). Three kinds of operations, corresponding the map scrolling, the radio tuning operation and the telephone number input operation are performed as a dual task (for about three minutes for each operation). Next, four levels of a navigation device operation condition are performed to provide an analysis object.

Experiment 1. Dual Task (CNTROL)

The DS driving and the stimulus detection are simultaneously performed, but the navigation device is not operated (for about twelve minutes).

Experiment 2. Triple Task (MAP)

The DS driving, the map scroll of the navigation device and the stimulus detection are simultaneously performed (for about twelve minutes).

Experiment 3. Triple Task (RADIO)

The DS driving, the radio channel tuning and the stimulus detection are simultaneously performed (for about twelve minutes).

Experiment 4. Triple Task (TEL)

The DS driving, the telephone number input of the navigation device and the stimulus detection are simultaneously performed (for about twelve minutes). Incidentally, order of the experiments 1 to 4 was adjusted by counterbalancing among the experimental participants. While the experiment was executed, in addition to a biological signal of the experimental participant, a behavior score of the stimulus detection task and a vehicle movement signal calculated by the DS were measured.

When each experiment, corresponding to each level, ends, a subjective workload was evaluated by NASA-TLX. The experiment was performed for two days. The first day was used as a practice day. Informed consent is given to the experimental participant. The experimental participant understood the experimental task and use of NASA-TLX, and got used to NASA-TLX. The second day was used to measure and obtain data.

2.4 Biological Measurement

The measured biological signal is the muscle potential. The muscle potential was measured by the active electrode (NIHON KOHDEN, NM-512G). The active electrode was pasted at an upper trapezius muscle of the right shoulder of the operator. A sampling frequency was set to 500 Hz. Incidentally, a body earth of the muscle potential was attached on an abdominal costal bone.

2.5 Data Analysis

In the experiment, it was detected whether the muscle potential changes according to a change of a mental load depending on difficulty level of the experimental tasks.

In this experiment, an analysis of variance was performed among the experimental participants in a condition where the experimental task corresponds to an independent variable and the RMS value of the muscle potential corresponds to a dependent variable. Incidentally, in order to remove an effect of an individual difference among the experimental participants, standard scores were generated among the experimental tasks of each of the experimental participant so that the biological signals were normalized among the experimental participants.

2.5.1 Muscle Potential Analysis

In this experiment, it was determined whether the muscle potential indicates a mental load such as the stress which the operator feels in the navigation device operation.

In the muscle potential analysis, the root mean square (RMS) is calculated from data of the muscle potential. One RMS value is calculated by moving average of 100 data points, provided by 50 data points before an arbitrary data point in a muscle potential data string and 50 data points after the arbitrary data point.

It is assumed that a person in the experimental participants with a small RMS mean value in all experimental tasks over an entire period does not use the trapezius muscle in a steering operation. Thus, it is assumed that the muscle potential of the person in the experimental participants is not likely to change according to the mental load.

Therefore, a threshold value is set according to the RMS mean value of all the experimental tasks among all the experimental participants, The RMS mean values of the eight experimental participants were smaller than the threshold value. Thus, results of eight participants were removed from the muscle potential evaluation.

The experimental participant operates the navigation device with his left hand and the steering with his right hand. If the muscle potential during the navigation operation is changed according to the kinds of the navigation operation tasks, it is considered that the mental load such as a time pressure or stress caused by the navigation device operation can be evaluated by the muscle potential.

Thus, in the present experiment, the RMS value of the muscle potential in the navigation device operation was compared within the four levels of the navigation device operation conditions. In the condition where the navigation device is not operated (corresponding to CONTROL in section 2.3), the RMS value of the muscle potential is sampled over the entire period. In the condition where the navigation device is operated (corresponding to MAP, RADIO or TEL in section 2.3), a period when the experimental participant operates only the navigation device is extracted from all experimental periods and the RMS value of the muscle potential corresponding to the period is analyzed. After the scores in the four levels of the navigation device operation condition were standardized in each of the experimental participants, a one-way analysis of variance was performed to the experimental task as a factor and Tukey's multiple comparison was performed.

In addition, a correlation between the muscle potential in the navigation device operation and an action indicator (Le., a missed rate in the stimulus detection task) and a correlation between the muscle potential and the subjective evaluation (i.e., WWL score in NASA-TLX) were calculated. It was determined whether it is appropriate to measure the muscle potential.

Incidentally, the missed rate is defined by dividing the number of the invalid trial by the sum of the number of the valid trial and the number of the invalid trial.

3. Analysis Result

(1) Comparison of Muscle Potential in Navigation Device Operation in Four Levels of Navigation Device Operation Condition

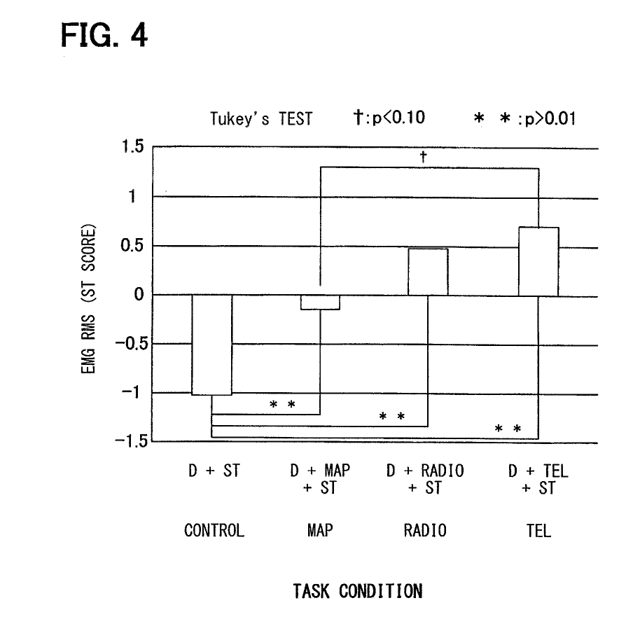

FIG. 4 illustrates a relationship between each of the navigation device control conditions and the muscle potential of the upper trapezius muscle of the right shoulder in the navigation device operation. The one-way analysis of variance showed that the navigation device operation condition has a main effect (F (3, 40)=13.92, p<0.01). As a result of Tukey's multiple comparison, the muscle potential in the navigation device operation condition (he., MAP, RADIO, or TEL) is significantly larger than the muscle potential in CONTROL (p<0.01). In addition, the muscle potential in TEL has slightly significantly larger than the muscle potential in MAP (p<0.10).

(2) Correlationship between Muscle Potential in Navigation Device Operation and Other Indicators

The muscle potential (the RMS value) in the navigation device operation was positively correlated with the action indicator (i.e., the missed rate in the stimulus detection task) (r=0.68, t (44)=5.97, p<0.01). The muscle potential (the RMS value) in the navigation device operation was positively correlated with the subjective evaluation (i.e., the WWL score in NASA-TLX) (r=0.65, t (40)=5.27, p<0.01).

In the above analysis, the number of samples is ten in a case where the correlation between the subjective evaluation and the muscle potential is calculated because one experimental participant did not fill the NASA-TLX partially.

4. Discussion

The muscle potential in the navigation device operation condition (MAP, RADIO or TEL) was larger than CONTROL. Thus, it is considered that the muscle potential significantly increases with an increase of the mental load by the navigation device operation or a physical load by a one-handed driving.

In addition, since it was observed that the muscle potential in TEL was slightly significantly larger than the muscle potential in MAP, it is suggested that the muscle potential indicates a change of the mental load according to a difference of the navigation device operations. Therefore, the present experiment may illustrate stress or anxiety feeling when a person operates a navigation device.

In addition, the correlation between the muscle potential in the navigation device operations and the action indicator was observed. The correlation between the muscle potential in the navigation device operations and the subjective evaluation was observed. Thus, it may be possible that the muscle potential reflects a variation of the mental load in the navigation device operation. It may be possible that the stimulus detection task method or NASA-TLX method indicates the mental load.

5. Conclusion

As described above, the muscle potential responds to a variation of the mental load. The muscle potential has correlation with the subjective evaluation or the action indicator. Thus, it is possible to use the muscle potential so as to evaluate the mental load.

### ADVANTAGES OF PRESENT DISCLOSURE

The stress evaluation apparatus 1 quantitatively measures the tension or strain of the operator in the trapezius muscle, which is not an agonist muscle for operating a steering wheel or the navigation device 7. According to the stress evaluation apparatus 1, it is possible to objectively estimate the mental load (including stress that the operator does not notice) which is caused by response to an external circumstance in driving or operation of the in-vehicle device or a carried-in device.

In the present embodiment, the user presentation process (at step S22) prohibits each of the operations, or informs that a predetermined operation is prohibited through the speaker 5 in a case where the operator feels the mental load to a hazardous degree. Thus, it is possible to prevent from performing a dangerous operation when the user feels stress.

Incidentally, in the present embodiment, the muscle potential of the trapezius muscle is measured in order to precisely measure the tension or the strain in driving associated with operation of a driving apparatus (e.g., a steering wheel, a gearshift), an in-vehicle device (e.g., a navigation device, a button), or a carried-in device (e.g., a mobile phone, a shaver). It is not limited to the trapezius muscle and it is possible to obtain a similar effect by measuring any muscle connecting the upper limb with a spine, any muscle connecting the upper limb with an anterior chest wall and a lateral chest wall.

Next, in the above evaluation process, the RMS value is calculated as strength information of the muscle potential of the trapezius muscle. The value of the integrated electromyogram (IEMG) may be used.

### Another Embodiment

In the above embodiment, in order to evaluate the stress in a right-hand vehicle, the active electrode 3 is pasted at a position where the muscle potential of the right trapezius muscle is measurable. In a case where a left-hand vehicle is used, the active electrode 3 may be pasted at a position where the muscle potential of a left trapezius muscle is measurable.

The processes at steps S16 to S20 in the evaluation process may be performed in the data center 9. The process for calculating the score relation information may be performed from a time when the operator pushes an operation start button to a time when the operator pushes an operation end button.

Incidentally, the evaluation process may only be performed in a case where the navigation device 7 obtains traffic information from external portion and a road shape is complicated or a traffic is crowded.

The evaluation process may be stopped temporarily in a backup or in a backward movement Furthermore, the evaluation process may be stopped temporarily by inputting, for example, blinker information when the vehicle turns to right or left.

In the above embodiment, the active electrode 3 may correspond to a detection sensor. Step S16 to step S18 may correspond to a calculation process portion or a calculation process step. Step S20 may correspond to an evaluation portion or an evaluation step. Step S22 may correspond to a presentation process portion or a presentation process step.

The detection step may correspond to a step for detecting the muscle potential using the detection sensor. A program executing the evaluation process in FIG. 3 may correspond to a stress evaluation program according to the present disclosure.

Incidentally, the present disclosure includes variations within a scope of the present disclosure described. Thus, it is not limited to the above described embodiment.

In the present disclosure, a stress evaluation apparatus is provided in various forms. For example, the stress evaluation apparatus may include a detection sensor, a calculation process portion and an evaluation portion. The detection sensor detects the muscle potential of either of the measuring object muscles of the operator. The measuring object muscles include a muscle connecting an upper limb and a spine, or a muscle that connects the upper limb and an anterior chest wall and that connects the upper limb a lateral chest wall.

In a case where the operator needs to integrally operate various devices, including the vehicle information device, with using the upper limb and when the operator has various stress factors such as an external driving circumstance, a long driving, or the like, the tension or the strains of the measuring object muscle reflects the stress when each of the operations is performed in driving. Thus, it is possible to objectively estimate the stress of the operator who performs each of the operations with various stress factors in a case where the muscle potential is detected.

In the stress evaluation apparatus, the strength information of the muscle potential of the measuring object muscle is calculated by the calculation process portion based on time series information of the muscle potential, detected by the detection sensor. The evaluation portion compares the strength information with a predetermined evaluation standard and evaluates the stress that the operator receives when the operator operates the in-vehicle device with the upper limb.

Therefore, according to the stress evaluation apparatus, it is possible to objectively evaluate the stress that the operator receives when the operator operates various operation devices, including the vehicle information device. Incidentally, the measuring object muscle is not the agonist muscle for moving the upper limb. The measuring object muscle may be a synergistic muscle and may include the muscle connecting the upper limb and the spine or the muscle that connects the upper limb and the anterior chest wall and that connects the upper limb the lateral chest wall as described above, preferably. The measuring object muscle may be the trapezius muscle.

The muscle connecting the upper limb and the spine includes the trapezius muscle, a latissimus dorsi muscle, a greater rhomboid muscle, a lesser rhomboid muscle, a levator scapulae muscle, for example. The muscle that connects the upper limb and the anterior chest wall and that connects the upper limb the lateral chest wall includes a greater pectoral muscle, a smaller pectoral muscle, a subclavian muscle, a anterior serratus muscle, for example (referring to Zusetsu Kin No Kinoukaibou 4^{th} edition Jul. 1, 2005, 11^{th} impression, Igaku-Shoin Ltd., John H. Warfel).

In addition, the evaluation standard may be calculated according to statistics (i.e., a mean value, a median value, a standard deviation or the like) of the standard scores of the RMS value of multiple persons. In this case, the multiple persons perform each of the operations with the operation devices that are operated by the upper limb, in advance. In addition, the evaluation standard may be set by an arbitrary manner.

Next, although the navigation device is considered as the in-vehicle device, the in-vehicle device includes the driving apparatus (e.g., a steering wheel, a gearshift), the in-vehicle device (e.g., a navigation device, a button) or the carried-in device (e.g., a mobile phone, a shaver).

Incidentally, the operation to the in-vehicle device includes, for example, a button operation, or a scroll operation. The stress evaluation apparatus may include a presentation process portion for executing the presentation process corresponding to the evaluation result in the evaluation portion.

In a case where a presentation is performed according to the result in the stress evaluation apparatus, even when the operator does not realize stress, the operator can objectively understand the stress which the operator receives. Thus, it is possible to facilitate that the vehicle is operated safely.

Incidentally, the presentation process may include a process by which a predetermined operation is prohibited, a process by which notice is provided to the external portion, or a process by which an alarm sound or an alarm display is produced. The presentation process may include a process by which a distance between vehicles increases or a process by which the vehicle is slowed down by a brake in a case where the distance between vehicles cab be controlled.

Incidentally, a stress evaluation program which executes the stress evaluation apparatus has advantages similar to the stress evaluation apparatus and therefore the explanation will be skipped.

While the present disclosure has been described with reference to embodiments thereof, it is to be understood that the disclosure is not limited to the embodiments and constructions. The present disclosure is intended to cover various modification and equivalent arrangements. In addition, while the various combinations and configurations, other combinations and configurations, including more, less or only a single element, are also within the spirit and scope of the present disclosure.

What is claimed is:

1. A stress evaluation apparatus comprising:
a detection sensor for detecting a muscle potential of a measuring object muscle of an operator;
a calculation process portion for receiving time series information of the muscle potential, which is detected by the detection sensor, and calculating strength information of the muscle potential based on the received time series information of the muscle potential; and
an evaluation portion for comparing the calculated strength information with a predetermined evaluation standard to make an evaluation of stress that the operator receives while an in-vehicle device is operated by the operator, wherein:
the measuring object muscle includes:
a muscle that connects an upper limb and a spine; or
another muscle that connects the upper limb and an anterior chest wall and that connects the upper limb and a lateral chest wall; and
the in-vehicle device is operated by the upper limb of the operator.

2. The stress evaluation apparatus according to claim 1, wherein
the measuring object muscle is a trapezius muscle.

3. The stress evaluation apparatus according to claim 1, wherein
the in-vehicle device is a navigation device.

4. The stress evaluation apparatus according to claim 1, further comprising
a presentation process portion for executing a presentation process for responding to a result of the evaluation made by the evaluation portion.

5. A non-transitory tangible computer readable storage medium storing a computer-executable program that causes a computer to perform a stress evaluation method, the method comprising:
detecting a muscle potential of a measuring object muscle of an operator by a detection sensor;
receiving time series information of the muscle potential detected by the detection sensor, and calculating strength information of the muscle potential based on the received time series information of the muscle potential; and
comparing the strength information with a predetermined evaluation standard to make an evaluation of stress that the operator receives when an in-vehicle device is operated by the operator, wherein:
the measuring object muscle includes:
a muscle that connects an upper limb and a spine; or
another muscle that connects the upper limb and an anterior chest wall and that connects the upper limb and a lateral chest wall; and
the in-vehicle device is operated by the upper limb of the operator.

6. The non-transitory tangible computer readable storage medium according to claim 5, wherein
the measuring object muscle is a trapezius muscle.

7. The non-transitory tangible computer readable storage medium according to claim 5, wherein
the in-vehicle device is a navigation device.

8. The non-transitory tangible computer readable storage medium according to claim 5, wherein the method further comprises
executing a presentation process for responding to a result of the evaluation made by the comparing.

9. A stress evaluation method comprising:
detecting a muscle potential of a measuring object muscle of an operator by a detection sensor;
receiving time series information of the muscle potential detected by the detection sensor, and calculating strength information of the muscle potential based on the received time series information of the muscle potential; and
comparing the strength information with a predetermined evaluation standard to make an evaluation of stress that the operator receives when an in-vehicle device is operated by the operator, wherein:
the measuring object muscle includes:
a muscle that connects an upper limb and a spine; or
another muscle that connects the upper limb and an anterior chest wall and that connects the upper limb and a lateral chest wall; and
the in-vehicle device is operated by the upper limb of the operator.

10. The stress evaluation apparatus according to claim 4, wherein
the presentation process includes prohibition of a predetermined operation, or notice that the predetermined operation has been prohibited based on the result of the evaluation made by the evaluation portion.
